(19) United States
(12) Patent Application Publication
(10) Pub. No.: US 2022/0000780 A1
(43) Pub. Date: Jan. 6, 2022

(54) NANODISCS FOR PREVENTING AND TREATING PATHOGEN INFECTIONS AND METHODS OF USE THEREOF

(71) Applicant: Mahmoud Nasr, Boston, MA (US)

(72) Inventor: Mahmoud Nasr, Boston, MA (US)

(21) Appl. No.: 17/291,332

(22) PCT Filed: Nov. 6, 2019
(86) PCT No.: PCT/US2019/060008

§ 371 Date: May 5, 2021

Related U.S. Application Data
(60) Provisional application No. 62/757,287, filed on Nov. 8, 2018.

(51) Int. Cl. A61K 9/127

(52) U.S. Cl. CPC A61K 9/1274; B82Y 5/00

(57) ABSTRACT

The invention provides compositions that contain pathogen-binding nanodiscs and methods of using such compositions to treat and prevent pathogen infections. The compositions include nanodiscs functionalized with receptors that bind a pathogen. Binding of a pathogen to the nanodiscs neutralizes the pathogen by interfering with one or more aspects of its reproductive cycle. The compositions and methods are useful for treating or preventing infections in the body and for disinfecting aqueous fluids that may contain pathogens.

### CROSS-REFERENCE TO RELATED APPLICATION

This application claims the benefit of, and priority to, U.S. Provisional Application No. 62/757,287, filed Nov. 8, 2018, the contents of which are incorporated herein by reference.

### FIELD OF THE INVENTION

The invention generally relates to compositions and methods for treating and preventing pathogen infections in the body and for disinfecting aqueous fluids.

### BACKGROUND

Infectious diseases account for three of the top ten causes of death worldwide and five of the top ten causes of death in low-income countries. The infectious diseases on those lists include lower respiratory infections, tuberculosis, diarrheal diseases, acquired immunodeficiency syndrome (AIDS), and malaria, which together kill more than seven million people each year. The economic burden of associated with infectious diseases is about $120 billion per year in the United States alone.

Although the last century has seen great advances in our ability to combat the viral and bacterial pathogens that cause most clinically significant infectious diseases, methods of treating and preventing infections still face serious limitations. Vaccines are excellent prophylactics against many infectious diseases, but they typically require weeks to months to provide immunity against the targeted pathogen. In addition, vaccines often take decades to develop, and there are currently no vaccines against several important pathogens, such as human immunodeficiency virus (HIV) and herpes viruses. Bacterial infections may be treated with antibiotics, but antibiotic resistance has increased over the last few decades, while few effective new antibiotics have reached the clinics. Antiviral agents that prevent viral replication inside the body have been developed for several viruses, but problems such as poor bioavailability and harmful side effects limit the utility of many such agents. Consequently, there is a lack of tools to fight many pathogens, and bacterial and viral infections continue to kill or sicken millions of people each year.

### SUMMARY

The invention provides a new approach to combating, preventing, and treating microorganism infections (e.g., viral or bacterial infections). Immunity to microbial infection is caused by a variety of specific and nonspecific mechanisms. Antibody neutralization is most effective when a microorganism (e.g., virus or bacteria) is present in large fluid spaces (e.g., serum) or on moist surfaces (e.g., the gastrointestinal and respiratory tracts). For example, antibodies can neutralize a virus by blocking virus-host cell interactions or recognizing viral antigens on virus-infected cells, which can lead to antibody-dependent cytotoxic cells (ADCC). In addition to antibodies, Cytotoxic T lymphocytes, natural killer (NK) cells and antiviral macrophages can recognize and kill virus-infected cells.

The invention leverages knowledge of how antibodies fight, prevent, and treat infections and provides nanodiscs incorporating a primary receptor of a microorganism (e.g., virus) that mimic antibody action to fight different infections. Particularly, the invention provides nanodiscs that have a plurality of receptors for one or more microorganisms (e.g., viruses), and uses those compositions to prevent/fight infection. Besides binding directly to a microorganism, the nanodiscs of the invention enhance phagocytosis upon microorganism binding. Consequently, spread of an infection will be effectively eliminated by low levels of circulating nanodisc.

Accordingly, the invention provides compositions that contain pathogen-binding nanodiscs and also provides for the use of such compositions to treat and prevent pathogen infections. In certain embodiments, the nanodiscs have multiple pathogen-binding receptors embedded in, or attached to, two-dimensional lipid bilayers that are, for example, up to 100 nm in diameter. Therefore, the receptor-containing nanodiscs facilitate binding of pathogen particles, such as bacterial cells or viral particles, by presenting receptors in a biophysical environment that resembles the plasma membrane of a target cell. Binding of pathogens to nanodiscs interferes with a pathogens' ability to attach to, enter, reproduce within, and exit from target cells. In addition, for some viruses, presentation of receptors in proximity to a lipid bilayer also triggers virus particles to inject their nucleic acid genomes across the bilayer in a futile attempt at infection, and separation of the viral nucleic acid from the remainder of the viral particle neutralizes the virus. Finally, because each nanodisc contains multiple receptors, the nanodiscs promote aggregation of pathogen particles, which triggers phagocytosis and degradation of the particles by macrophages. Thus, administration of the nanodiscs containing receptors to a particular virus, bacterium, or other pathogen protects a person from the harmful effects of that pathogen.

The methods of the invention are broadly applicable for treatment and prevention of infection by a wide variety of pathogens. Because the methods rely on the incorporation of multiple copies of a pathogen-binding receptor, such as a cell-surface molecule or antibody, into the nanodisc, they are not restricted by the particularities of the reproductive cycle of a given pathogenic microorganism. Thus, the methods can be easily adapted to combat infection by any pathogen of interest.

The methods are also useful to fight infections before or after exposure to a pathogen. Upon administration, the receptor-containing nanodiscs circulate freely in the blood and interstitial fluids, so they bind to targeted pathogen particles already present in the body as well as those contracted during the circulating lifespan of the nanodiscs. Moreover, the pathogen-aggregating properties of the receptor-containing nanodiscs promote the body's own immune response, which complements inhibition of pathogen replication by intrinsic mechanisms, such as prevention of pathogen particles from entering or exiting target cells.

The invention also provides methods of disinfecting aqueous fluids, such as beverages, body fluids, and medical supplies, using receptor-containing nanodiscs. According to the same principles described above, addition of receptor-containing nanodiscs to aqueous fluids promotes binding and aggregation of targeted pathogen particles contained in the fluids. Consequently, the pathogen particles are disabled from infecting individuals who come in contact with treated fluids.

In an aspect, the invention provides methods of treating or preventing a pathogen infection in a subject. The methods include providing to a subject a composition containing multiple nanodiscs functionalized with a plurality of receptors that specifically bind a pathogen. The nanodiscs specifically bind the pathogen or a pathogen-specific marker on an infected cell to prevent the pathogen from interacting with a target cell or inhibit one or more processes associated with pathogen replication in the target cell, thereby treating or preventing a pathogen infection in a subject.

The nanodiscs may circulate in the blood and/or interstitial fluid of the subject. The nanodiscs may promote aggregation of the pathogen via binding of the pathogen to receptors on one or more nanodiscs. Aggregation may reduce the number of circulating pathogens. Aggregation may facilitate or induce phagocytosis of the pathogen. Aggregation may prevent the pathogen from interacting with the target cell.

The pathogen may be any type of a pathogen. Preferably, the pathogen is a microorganism, such as a bacterium, virus, fungus, or plasmodium.

Binding of a pathogen, such as a virus, to one or more nanodiscs may inhibit a step in virus replication. For example, binding may inhibit one or more of attachment or adsorption of the virus to the surface of the target cell, penetration of the virus into the target cell, and uncoating of the virus within the target cell.

Binding of a pathogen-specific marker to one or more nanodiscs on the surface of an infected cell may inhibit budding of the pathogen, such as a virus, from the infected cell.

The nanodiscs of the composition may all be of the same size. The nanodiscs of the composition may be of different sizes. The nanodiscs may have diameters of from about 6 nm to about 100 nm, from about 10 nm to about 90 nm, from about 20 nm to about 80 nm, from about 30 nm to about 70 nm, from about 40 nm to about 60 nm, about 10 nm, about 20 nm, about 30 nm, about 40 nm, about 50 nm, about 60 nm, about 70 nm, about 80 nm, about 90 nm, or about 100 nm.

The nanodiscs may have any two-dimensional shape. For example, the nanodiscs may be circular, rectangular, triangular, pentagonal, hexagonal, heptagonal, or octagonal.

The nanodiscs may contain a belt, i.e., a molecular structure that forms a two-dimensional boundary that encloses the lipid bilayer within the nanodisc. The belt may be an amphipathic molecule or complex of molecules. The belt may contain one or more nucleic acids, such as RNA or DNA, proteins, polypeptides, peptides, a polymer, or any combination thereof. The protein or polypeptide may include portions of apolipoprotein A1 (apoA1). The nucleic acid may include DNA barrels assembled by scaffolded DNA origami. The belt may be circularized, i.e., it may have a continuous series of covalent bonds that circumscribes the lipid bilayer. The belt may be uncircularized.

The nanodiscs may contain any suitable amphipathic lipid. For example, the nanodiscs may contain one or more of 1-palmitoyl-2-oleoyl-sn-glycero-3-phosphocholine (POPC), 1-palmitoyl-2-oleoyl-sn-glycero-3-phosphoglycerol (POPG), 1,2-dimyristoyl-sn-glycero-3-phosphocholine (DMPC), or 1,2-Dimyristoyl-sn-glycero-3-phospholglycerol (DMPG). The nanodiscs may contain a mixture of lipids. The mixture may contain a defined molar ratio of specific lipids. For example, that nanodiscs may contain a 3:2 mixture of POPC:POPG or a 3:1 mixture of DMPC:DMPG. The lipid bilayer of the nanodiscs may be substantially free of detergent.

The receptor may be any molecule that binds to a pathogen. For example, the receptor may be a protein, polypeptide, protein complex, antibody, nucleic acid, carbohydrate, or combination or portion thereof. The receptor may be embedded in the lipid bilayer via a transmembrane domain. The receptor may be linked to the lipid bilayer via covalent attachment to a lipid.

One or more nanodiscs may have two or more sets of receptors in which different sets are specific for different pathogens. One or more nanodiscs may have two or more sets of receptors in which different sets are specific for the same pathogen. One or more nanodiscs may have multiple copies of a single receptor.

The composition may include two or more sets of nanodiscs in which different sets have different receptors specific for different pathogens. The composition may include two or more sets of nanodiscs in which different sets have different receptors specific for the same pathogen. The composition may include a single set of nanodiscs in which each nanodisc has multiple copies of a single receptor.

In embodiments in which the compositions have multiple sets of nanodiscs, the nanodiscs of the different sets may be present in the same amount, or the nanodiscs of the different sets may be present in different amounts.

In embodiments in which the compositions have multiple sets of nanodiscs, the nanodiscs of the different sets may differ by one or more structural element described above, such as shape, size, bilayer composition, or belt composition.

In another aspect, the invention provides compositions containing multiple nanodiscs in which each nanodisc is functionalized with multiple receptors that specifically bind a pathogen.

The nanodiscs may have any structural element described above. For example, the nanodiscs of the composition may have any size, shape, belt, lipid bilayer, or receptor described above.

One or more nanodiscs may have two or more sets of receptors in which different sets are specific for different pathogens. One or more nanodiscs may have two or more sets of receptors in which different sets are specific for the same pathogen. One or more nanodiscs may have multiple copies of a single receptor.

The composition may include two or more sets of nanodiscs in which different sets have different receptors specific for different pathogens. The composition may include two or more sets of nanodiscs in which different sets have different receptors specific for the same pathogen. The composition may include a single set of nanodiscs in which each nanodisc has multiple copies of a single receptor.

In embodiments in which the compositions have multiple sets of nanodiscs, the nanodiscs of the different sets may be present in the same amount, or the nanodiscs of the different sets may be present in different amounts.

In embodiments in which the compositions have multiple sets of nanodiscs, the nanodiscs of the different sets may differ by one or more structural element described above, such as shape, size, bilayer composition, or belt composition.

The pathogen may be any pathogen, such as any of those described above.

In another aspect, the invention provides methods of disinfecting an aqueous medium by adding to an aqueous medium a composition containing multiple nanodiscs in which each nanodisc is functionalized with multiple receptors that specifically bind a pathogen and in which the nanodiscs specifically bind the pathogen to prevent the pathogen from interacting with a target cell, inhibit pathogen replication in the target cell, or both, thereby disinfecting the aqueous medium.

The pathogen may be any pathogen, such as any of those described above.

The aqueous medium may be any aqueous fluid. The aqueous medium may be synthetic growth medium. The aqueous medium may be a biological fluid sample. The biological fluid may be blood, plasma, serum, semen, sputum, saliva, or milk. The aqueous medium may be a solution or suspension that will be contacted with, or transferred into, the body of a subject

The nanodiscs may have any structural element described above. For example, the nanodiscs of the composition may have any size, shape, belt, lipid bilayer, or receptor described above. The nanodiscs may include multiple sets of receptors, as described above.

The composition may include two or more sets of nanodiscs, as described above. The two or more sets may have different receptors, as described above. The two or more sets may be present in the same amount or in different amounts, as described above.

### BRIEF DESCRIPTION OF THE DRAWINGS

FIG. 1 shows a nanodisc according to an embodiment of the invention.

FIG. 2 illustrates a mechanism of extracellular neutralization of virus by nanodiscs according to an embodiment of the invention.

FIG. 3 illustrates additional mechanisms of neutralization of virus by nanodiscs according to embodiments of the invention.

### DETAILED DESCRIPTION

The invention provides methods for treating and preventing infections using pathogen-binding nanodiscs to neutralize pathogens in the blood and other body fluids. The nanodiscs contain lipid bilayers that are functionalized with multiple copies of a pathogen receptor.

Consequently, the nanodiscs bind to pathogens, such as viruses, to prevent their reproduction in target cells by one or more of a variety of mechanisms. Due to the size of the nanodiscs and their capacity to bind multiple pathogen particles simultaneously, the nanodiscs also induce aggregation of virus particles and bacterial cells, which facilitates their phagocytosis by macrophages. Thus, the nanodiscs also decrease infectivity of pathogens in a target cell-independent manner and facilitate the body's immune response to pathogens.

The compositions and methods of the invention are useful for preventing infection in a variety of contexts. The methods are applicable for a wide range of pathogens because they interfere with processes that are common to many pathogens. The methods are effective both before and after an individual has been exposed to a pathogen, and their prophylactic use does not require a prolonged waiting period prior to risk of exposure to the pathogen. In addition, the methods can neutralize pathogens outside of the body and thus can be used to disinfect blood products, medical supplies, foodstuffs, and other items that may carry pathogens.

### Nanodiscs

The compositions and methods of the invention include nanodiscs. Generally, nanodiscs are structures having lipid bilayers circumscribed by a belt or border that contains an amphipathic molecular structure. Due to the amphipathic nature of the lipids and the belt structure, nanodiscs are stable in aqueous media. As is described in more detail below, nanodiscs may assume a variety of two-dimensional shapes and sizes but generally have diameters of from about 1 nm to about 1000 nm. Nanodiscs and methods of making nanodiscs are known in the art and described in, for example, Nasr, M. L., et al., Covalently circularized nanodiscs for studying membrane proteins and viral entry, Nat Methods, 2017 January, 14(1): 49-52, doi:10.1038/nmeth.4079; Zhao, Z., et al., DNA-Corralled Nanodiscs for the Structural and Functional Characterization of Membrane Proteins and Viral Entry, J. Am. Chem. Soc. 2018, 140, 10639-10643, DOI:10.1021/jacs.8b04638; Yasuhara, K., et al., Spontaneous Lipid Nanodisc Formation by Amphiphilic Polymethacrylate Copolymers, J Am Chem Soc. 2017 December 27; 139(51): 18657-18663, doi:10.1021/jacs.7b10591; International Publication No. WO 2018/017442; and co-pending International Application No. PCT/US18/32862, the contents of each of which are incorporated herein by reference.

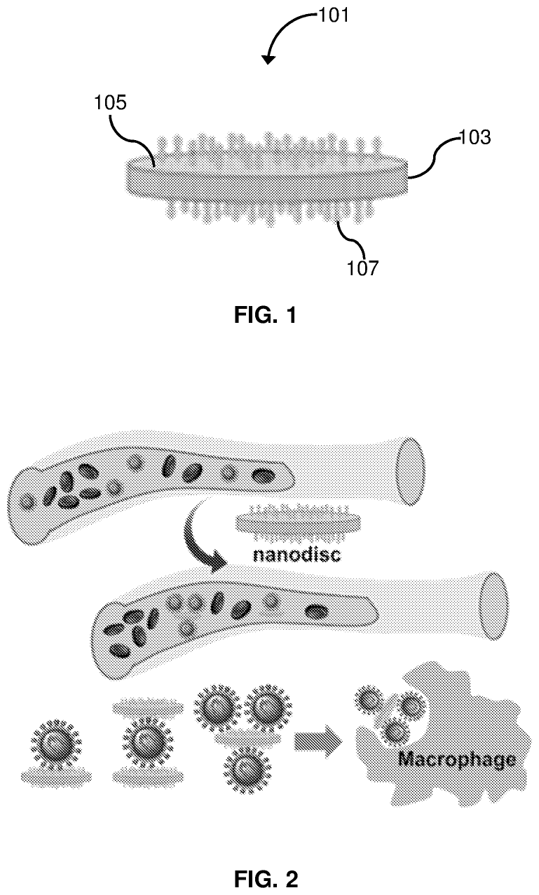

FIG. 1 shows a nanodisc 101 according to an embodiment of the invention. The nanodisc 101 includes a belt 103 that circumscribes a lipid bilayer 105. The nanodisc 101 is functionalized with multiple receptors 107 that bind a pathogen.

Pathogen Receptors

Any molecule of moiety may be used as a pathogen receptor. For example and without limitation, the receptor may be a protein, polypeptide, protein complex, antibody, nucleic acid, carbohydrate, or combination or portion thereof.

The receptor may be or may contain a receptor or portion of a receptor for a virus. Viruses typically bind to one or more naturally-occurring proteins or glycoproteins on the surface of cells. For example and without limitation, the molecules known to function as receptors or co-receptors for particular viruses include the following: CD4, CCR5, and CXCR4 for human immunodeficiency virus (HIV); CD81, SR-B1, claudin-1, and occludin for hepatitis C virus (HCV); CD55 and CAR for cocksackie virus B; αvβ3/αvβ5 integrins for adenovirus; CD155 for poliovirus; ICAM-1, LDLR, and CDHR3 for rhinoviruses; and sialic acid for influenza. Many receptors are known in the art and are described, for example, Grove J., and Marsh, M., The cell biology of receptor-mediated virus entry, J. Cell Biol. Vol. 195 No. 7 1071-1082, doi/10.1083/jcb.201108131, the contents of which are incorporated herein by reference.

In some case, interactions between virus particles and target cells are initially mediated by non-specific electrostatic interactions between viral proteins and glycolipids or glycoprotein attachment factors on the cell surface, such as heparan sulfate proteoglycan. Thus, the nanodiscs may include additional non-receptor attachment factors, such as heparan sulfate proteoglycan, to facilitate interaction between the nanodiscs and virus particles.

Alternatively or additionally, the receptor may be an antibody. An antibody may be a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, a polyaminoacid, or the like.

The receptor may be attached to the nanodisc by any suitable means. For example, the receptor may be embedded in the lipid bilayer via a transmembrane domain. The transmembrane domain may be a native component of the receptor, or it may be a recombinant portion added from another polypeptide. The receptor may be linked to the lipid bilayer via covalent or non-covalent attachment to a lipid. One example of a non-covalent linkage conjugation to lipids via nitrilotriacetic acid-nickel, as described in Nasr, M. L., et al., Covalently circularized nanodiscs for studying membrane proteins and viral entry, Nat Methods, 2017 January, 14(1): 49-52, doi:10.1038/nmeth.4079; and International Publication No. WO 2018/017442, the contents of each of which are incorporated herein by reference.

According to embodiments of the invention, individual nanodiscs include multiple receptor molecules. A nanodisc may contain a single receptor that is present in multiple copies on the nanodisc. Alternatively, the nanodisc may contain two or more different receptors, each of which is present in multiple copies on the nanodisc. In nanodiscs that include multiple receptors, the different receptors may be receptors for different pathogens, e.g., different viruses. Alternatively or additionally, in nanodiscs that include multiple receptors, the different receptors may be different receptors for the same pathogen. As indicated above, some viruses bind to more than one cellular protein to gain entry into a target cell. For example, CD81, SR-B1, claudin-1, and occludin are all necessary for entry of HCV. Thus, the nanodisc may contain two or more receptors for the same pathogen to mimic more closely the surface of a target cell that the pathogen infects.

Shape and Size of Nanodiscs

Nanodiscs may have any suitable two-dimensional shape. For example and without limitation, the nanodiscs may be circular, rectangular, triangular, pentagonal, hexagonal, heptagonal, or octagonal. The two-dimensional shape of a nanodisc is influenced by the structure of its belt. Nanodiscs of various shapes are described in, for example, Nasr, M. L., et al., Covalently circularized nanodiscs for studying membrane proteins and viral entry, Nat Methods, 2017 January, 14(1): 49-52, doi:10.1038/nmeth.4079; and International Publication No. WO 2018/017442, the contents of each of which are incorporated herein by reference.

Nanodiscs may have a diameter, i.e., a measurement across the two-dimensional structure, of from about 6 nm to about 100 nm, from about 10 nm to about 90 nm, from about 20 nm to about 80 nm, from about 30 nm to about 70 nm, from about 40 nm to about 60 nm, about 10 nm, about 20 nm, about 30 nm, about 40 nm, about 50 nm, about 60 nm, about 70 nm, about 80 nm, about 90 nm, about 100 nm, about 150 nm, about 200 nm, about 300 nm, about 400 nm, about 500 nm, about 600 nm, about 800 nm, or about 1000 nm.

The diameter of a nanodisc may be 10-200 nm. In some embodiments, the nanodisc has a diameter of 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, or 200 nm. In some embodiments, the nanodisc has a diameter of 20-30 nm, 20-40 nm, 20-50 nm, 20-60 nm, 20-70 nm, 20-80 nm, 20-90 nm, 20-100 nm, 20-150 nm, 20-200 nm, 30-40 nm, 30-50 nm, 30-60 nm, 30-70 nm, 30-80 nm, 30-90 nm, 30-100 nm, 30-150 nm, or 30-200 nm. In some embodiments, the nanodisc has a diameter of greater than 200 nm or less than 10 nm.

In some embodiments, the diameter of the nanodisc is greater than about 6 nm, greater than about 15 nm, greater than about 50 nm, or greater than about 80 nm. In some embodiments, the diameter of the nanodisc is from about 6 nm to about 80 nm, from about 15 nm to about 80 nm, or from about 50 nm to about 80 nm.

Belts of Nanodiscs

The belt of the nanodisc may be an amphipathic molecule or complex of molecules. The belt may contain one or more nucleic acids, such as RNA or DNA, proteins, polypeptides, peptides, a polymer, or any combination thereof. The protein or polypeptide may include portions of apolipoprotein A1 (apoA1). The nucleic acid may include DNA barrels assembled by scaffolded DNA origami. The belt may be circularized, i.e., it may have a continuous series of covalent bonds that circumscribes the lipid bilayer. The belt may be uncircularized.

Nanodisc belts may consist of or contain belt proteins. Typically, belt proteins are amphipathic alpha helical proteins that bind the phospholipid bilayer periphery, surrounding the bilayer. Belt proteins generally have hydrophobic faces that associate with the nonpolar interior of the phospholipid bilayer and hydrophilic faces that interact with the aqueous exterior environment. In some embodiments, the belts do not completely encircle the nanodisc. In other embodiments, the belts do completely encircle the nanodisc. In some embodiments, a nanodisc is associated with 1, 2, 3, 4, 5, 6, 7, or more proteins or polypeptides. Belt proteins may be naturally occurring (for example, apolipoproteins A, (A-I and A-II), B, C, D, E, and H) or engineered (for example, using recombinant technologies). Examples of belt proteins include, but are not limited to MSP1, MSP1TEV, MSP1D1, MSP1D1-D73C, MSP1D1(-), MSP1E1, MSP1E1D1, MSP1E2, MSP1E2D1, MSP1E3, MSP1E3D1, MSP1E3D1-D73C, MSP1D1-22, MSP1D1-33, MSP1D1-44, MSP2, MSP2N2, MSP2N3, MSP1FC, MSP1FN. Other examples of belt proteins are known in the art and described in, for example, International Publication No. WO 2018/017442; and co-pending International Application No. PCT/US18/32862, the contents of each of which are incorporated herein by reference. Nanodiscs may be composed of belt protein variants. In some embodiments, the belt protein variants comprise introduced unnatural amino acids, for example, cysteine derivatives. Other examples of unnatural amino acids include, but are not limited to, alanine derivatives, alicyclic amino acids, arginine derivatives, aromatic amino acids, asparagine derivatives, aspartic acid derivatives, beta-amino acids, 2,4-diaminobutyric acid (DAB), 2,3-diaminopropionic acid, glutamic acid derivatives, glutamine derivatives, glycine derivatives, homo-amino acids, isoleucine derivatives, leucine derivatives, linear core amino acids, lysine derivatives, methionine derivatives, N-methyl amino acids, norleucine derivatives, norvaline derivatives, ornithine derivatives, penicillamine derivatives, phenylalanine derivatives, phenylglycine derivatives, proline derivatives, pyroglytamine derivatives, serine derivatives, threonine derivatives, tryptophan derivatives, tyrosine derivatives, and valine derivatives. Other examples of unnatural amino acids are known. For example, Nanodisc Width 11 nm (NW11) constructs may be used to engineer covalently circularized nanodiscs (see, e.g., Nasr, M. L. et al. Nature Methods, 14(1): 49-54, 2017, incorporated herein by reference in its entirety). The N and C termini of NW11 variants are covalently linked to each other to form a stable barrier. These belt protein constructs contain the consensus sequence recognized by sortase A (LPGTG; SEQ ID NO: 2) near the C terminus and a single glycine residue at the N terminus (after TEV cleavage). The presence of these two sites ensures covalent linkage between the N and C termini of a protein while still conserving the function to form lipid nanodiscs. Belt proteins and their use in construction of nanodiscs are described in, for example, co-pending International Application No. PCT/US18/32862, the contents of which are incorporated herein by reference.

Nanodisc belts may consist of or contain nucleic acids. Nucleic acid belts may be nucleic acid nanostructures, i.e., engineered nanostructures (e.g., having a size of less than 1 μm) assembled from nucleic acids comprising nucleotide domains (regions) that hybridize to each other. Nucleic acid nanostructures may be assembled from a plurality of different nucleic acids (e.g., single-stranded nucleic acids, also referred to as single-stranded tiles, or SSTs (see, e.g., Wei, B. et al. Nature, 485, 623-626, 2012, incorporated herein by reference). For example, a nucleic acid nanostructure may be assembled from at least 10, at least 20, at least 30, at least 40, at least 50, at least 60, at least 70, at least 80, at least 90 or at least 100 nucleic acids. In some embodiments, a nucleic acid nanostructure is assembled from at least 100, at least 200, at least 300, at least 400, at least 500, or more, nucleic acids. The term “nucleic acid” encompasses “oligonucleotides,” which are short, single-stranded nucleic acids (e.g., DNA) having a length of 10 nucleotides to 200 nucleotides (also referred to in the art as “staple strands”). In some embodiments, an oligonucleotide has a length of 10 to 20 nucleotides, 10 to 30 nucleotides, 10 to 40 nucleotides, 10 to 50 nucleotides, 10 to 60 nucleotides, 10 to 70 nucleotides, 10 to 80 nucleotides, 10 to 90 nucleotides, 10 to 100 nucleotides, 10 to 150 nucleotides, or 10 to 200 nucleotides. In some embodiments, an oligonucleotide has a length of 20 to 50, 20 to 75 or 20 to 100 nucleotides. In some embodiments, an oligonucleotide has a length of 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190 or 200 nucleotides.

In some embodiments, a nucleic acid nanostructure is assembled from single-stranded nucleic acids, double-stranded nucleic acids, or a combination of single-stranded and double stranded nucleic acids (e.g., includes an end terminal single-stranded overhang).

Nucleic acid nanostructures may be assembled from a plurality of heterogeneous nucleic acids (e.g., oligonucleotides). “Heterogeneous” nucleic acids may differ from each other with respect to nucleotide sequence. For example, in a heterogeneous plurality that includes nucleic acids A, B, and C, the nucleotide sequence of nucleic acid A differs from the nucleotide sequence of nucleic acid B, which differs from the nucleotide sequence of nucleic acid C. Heterogeneous nucleic acids may also differ with respect to length and chemical compositions (e.g., isolated vs. synthetic).

The fundamental principle for designing self-assembled nucleic acid structures (e.g., nucleic acid nanostructures) is that sequence complementarity in nucleic acid strands is encoded such that, by pairing up complementary segments, the nucleic acid strands self-organize into a predefined nanostructure under appropriate physical conditions. From this basic principle (see, e.g., Seeman N. C. J. Theor. Biol. 99: 237, 1982, incorporated by reference herein), researchers have created diverse synthetic nucleic acid structures (e.g., nucleic acid nanostructures) (see, e.g., Seeman N. C. Nature 421: 427, 2003; Shih W. M. et al. Curr. Opin. Struct. Biol. 20: 276, 2010, each of which is incorporated by reference herein). Examples of nucleic acid (e.g., DNA) nanostructures, and methods of producing such structures, that may be used in accordance with the present disclosure are known and include, without limitation, lattices (see, e.g., Winfree E. et al. Nature 394: 539, 1998; Yan H. et al. Science 301: 1882, 2003; Yan H. et al. Proc. Natl. Acad. of Sci. USA 100; 8103, 2003; Liu D. et al. J. Am. Chem. Soc. 126: 2324, 2004; Rothemund P. W. K. et al. PLoS Biology 2: 2041, 2004, each of which is incorporated by reference herein), ribbons (see, e.g., Park S. H. et al. Nano Lett. 5: 729, 2005; Yin P. et al. Science 321: 824, 2008, each of which is incorporated by reference herein), tubes (see, e.g., Yan H. Science, 2003; P. Yin, 2008, each of which is incorporated by reference herein), finite two-dimensional and three dimensional objects with defined shapes (see, e.g., Chen J. et al. Nature 350: 631, 1991; Rothemund P. W. K., Nature, 2006; He Y. et al. Nature 452: 198, 2008; Ke Y. et al. Nano. Lett. 9: 2445, 2009; Douglas S. M. et al. Nature 459: 414, 2009; Dietz H. et al. Science 325: 725, 2009; Andersen E. S. et al. Nature 459: 73, 2009; Liedl T. et al. Nature Nanotech. 5: 520, 2010; Han D. et al. Science 332: 342, 2011, each of which is incorporated by reference herein), and macroscopic crystals (see, e.g., Meng J. P. et al. Nature 461: 74, 2009, incorporated by reference herein).

Examples of nucleic acid (e.g., DNA) nanostructures include, but are not limited to, DNA origami structures, in which a long scaffold strand (e.g., at least 500 nucleotides in length) is folded by hundreds (e.g., 100, 200, 200, 400, 500 or more) of short (e.g., less than 200, less than 100 nucleotides in length) auxiliary staple strands into a complex shape (Rothemund, P. W. K. Nature 440, 297-302 (2006); Douglas, S. M. et al. Nature 459, 414-418 (2009); Andersen, E. S. et al. Nature 459, 73-76 (2009); Dietz, H. et al. Science 325, 725-730 (2009); Han, D. et al. Science 332, 342-346 (2011); Liu, W. et al. Angew. Chem. Int. Ed. 50, 264-267 (2011); Zhao, Z. et al. Nano Lett. 11, 2997-3002 (2011); Woo, S. & Rothemund, P. Nat. Chem. 3, 620-627 (2011); Toning, T. et al. Chem. Soc. Rev. 40, 5636-5646 (2011), incorporated by reference herein). Staple strands are complementary to and bind to two or more noncontiguous regions of a scaffold strand.

In some embodiments, a scaffold strand is 100-10000 nucleotides in length. In some embodiments, a scaffold strand is at least 100, at least 500, at least 1000, at least 2000, at least 3000, at least 4000, at least 5000, at least 6000, at least 7000, at least 8000, at least 9000, or at least 10000 nucleotides in length. The scaffold strand may be naturally occurring or non-naturally occurring. In some embodiments, a single-stranded nucleic acid for assembly of a nucleic acid nanostructure has a length of 500 base pairs to 10 kilobases, or more. In some embodiments, a single-stranded nucleic acid for assembly of a nucleic acid nanostructure has a length of 4 to 5 kilobases, 5 to 6 kilobases, 6 to 7 kilobases, 7 to 8 kilobases, 8 to 9 kilobases, or 9 to 10 kilobases. Staple strands are typically shorter than 200 nucleotides in length; however, they may be longer or shorter depending on the application and depending upon the length of the scaffold strand (a staple strand is typically shorter than the scaffold strand). In some embodiments, a staple strand may be 15 to 100 nucleotides, or 15 to 200 nucleotides, in length.

In some embodiments, a staple strand is 25 to 50 nucleotides in length.

In some embodiments, a nucleic acid nanostructure may be assembled in the absence of a scaffold strand (e.g., a scaffold-free structure). For example, a number of oligonucleotides (e.g., less than 200 nucleotides or less than 100 nucleotides in length) may be assembled to form a nucleic acid nanostructure.

In some embodiments, a nucleic acid nanostructure may be assembled to form a self-limiting ring structure referred to herein as a ‘DNA-arena-scaffolded lipid nanodisc’. These assembled structures involve the linking of multiple copies of DNA origami heterodimers into a pre-determined, defined shape (e.g., a cylinder/barrel). Each DNA origami heterodimer includes two distinct DNA origami units linked by DNA hybridization at low magnesium concentration to form a V-shaped heterodimer. In some embodiments, the diameter of this assembled nanostructure is 200-1000 nm. In some embodiments, a cylindrical nucleic acid nanostructure has a diameter of 200-300 nm, 200-400 nm, 200-500 nm, 200-600 nm, 200-700 nm, 200-800 nm, 200-900 nm, or 200-1000 nm. In some embodiments, the diameter of this assembled nanostructure is greater than 1000 nm. some embodiments, the diameter of this assembled nanostructure is less than 200 nm.

In some embodiments, a nucleic acid nanostructure may be asymmetric with respect to lipid distribution. The incorporation of protein scramblases and flippases such as ABCA1, ABCA4 and ABCA7 into the lipid bilayer of the nanostructure allows for maintenance of the asymmetry.

In some embodiments, a nucleic acid nanostructure is assembled from single-stranded tiles (SSTs) (see, e.g., Wei B. et al. Nature 485: 626, 2012, incorporated by reference herein) or nucleic acid “bricks” (see, e.g., Ke Y. et al. Science 388:1177, 2012; International Publication Number WO 2014/018675 A1, published Jan. 30, 2014, each of which is incorporated by reference herein). For example, single-stranded 2- or 4-domain oligonucleotides self-assemble, through sequence-specific annealing, into two- and/or three-dimensional nanostructures in a predetermined (e.g., predicted) manner. As a result, the position of each oligonucleotide in the nanostructure is known. In this way, a nucleic acid nanostructure may be modified, for example, by adding, removing or replacing oligonucleotides at particular positions. The nanostructure may also be modified, for example, by attachment of moieties, at particular positions. This may be accomplished by using a modified oligonucleotide as a starting material or by modifying a particular oligonucleotide after the nanostructure is formed. Therefore, knowing the position of each of the starting oligonucleotides in the resultant nanostructure provides addressability to the nanostructure.

Other methods for assembling nucleic acid nanostructures are known in the art, any one of which may be used herein. Such methods are described by, for example, Bellot G. et al., Nature Methods, 8: 192-194 (2011); Liedl T. et al., Nature Nanotechnology, 5: 520-524 (2010); Shih W. M. et al., Curr. Opin. Struct. Biol., 20: 276-282 (2010); Ke Y. et al., J. Am. Chem. Soc., 131: 15903-08 (2009); Dietz H. et al., Science, 325: 725-30 (2009); Hogberg B. et al., J. Am. Chem. Soc., 131: 9154-55 (2009); Douglas S. M. et al., Nature, 459: 414-418 (2009); Jungmann R. et al., J. Am. Chem. Soc., 130: 10062-63 (2008); Shih W. M., Nature Materials, 7: 98-100 (2008); and Shih W. M., Nature, 427: 618-21 (2004), each of which is incorporated herein by reference in its entirety.

A nucleic acid nanostructure may be assembled into one of many defined and predetermined shapes, including without limitation a cylinder (barrel), a capsule, a hemi-sphere, a cube, a cuboidal, a tetrahedron, a cylinder, a cone, an octahedron, a prism, a sphere, a pyramid, a dodecahedron, and a tube. A nucleic acid nanostructure may be a geometric shape (easily recognizable, e.g., circle, triangle, rectangle, etc.) or may be an abstract shape (e.g., free-form, non-geometric curves, random angles, and/or irregular lines). It should be understood that a nucleic acid nanostructure is distinct from condensed nucleic acid (e.g., DNA having a solid or dense core) and may have a void volume (e.g., it may be partially or wholly hollow). In some embodiments, the void volume may be at least 10%, at least 15%, at least 20%, 25%, at least 30%, at least 35%, at least 40%, at least 45%, at least 50%, at least 55%, at least 60%, at least 65%, at least 70%, at least 75%, at least 80%, at least 85%, at least 90%, at least 91% at least 92%, at least 93%, at least 94%, at least 95%, at least 96%, at least 97%, at least 98%, at least 99%, or more of the volume of the nanostructure. The “void volume” of a nucleic acid nanostructure is the cumulative empty space (space not occupied by nucleic acid) within a nucleic acid nanostructure.

Nucleic acid nanostructures may comprise deoxyribonucleic acid (DNA), ribonucleic acid (RNA), modified DNA, modified RNA, peptide nucleic acid (PNA), locked nucleic acid (LNA), or any combination thereof. In some embodiments, a nucleic acid nanostructure is a DNA nanostructure. In some embodiments, a DNA nanostructure consists of DNA. A cylindrical nucleic acid nanostructure is a nucleic acid nanostructure that forms an exterior surface and an interior compartment (having an interior surface). A cylindrical nucleic acid (e.g., DNA) nanostructure may be comprised, for example, of one or more smaller stacked cylindrical nanostructured (e.g., each with two open ends). In some embodiments, a cylindrical nucleic acid nanostructure comprises at least two or at least three smaller (e.g., shorter) cylindrical nanostructures linked together to form one large cylinder. An entire cylindrical nucleic acid nanostructure, in some embodiments, may be made using a single (or two or three) long scaffold strand and shorter staple strands (e.g., using the DNA origami method). Other nucleic acid nanostructure assembly methods may be used and are described elsewhere herein. Generally, cylindrical nanostructures have an internal void volume that can be calculated by the formula: V=πr²a, where r is the radius of the cylinder and a is the height of the cylindrical part. The surface area formula is SA=2πra+2πr².

The diameter (e.g., inner diameter) of a cylindrical nucleic acid nanostructure may be, for example, 10-200 nm. In some embodiments, a cylindrical nucleic acid nanostructure has a diameter of 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, or 200 nm. In some embodiments, a cylindrical nucleic acid nanostructure has a diameter of 20-30 nm, 20-40 nm, 20-50 nm, 20-60 nm, 20-70 nm, 20-80 nm, 20-90 nm, 20-100 nm, 20-150 nm, 20-200 nm, 30-40 nm, 30-50 nm, 30-60 nm, 30-70 nm, 30-80 nm, 30-90 nm, 30-100 nm, 30-150 nm, or 30-200 nm. In some embodiments, a cylindrical nucleic acid nanostructure has a diameter of greater than 200 nm or less than 10 nm.

Nanodisc belts may consist of or contain synthetic polymers, such as polymethacrylate. The use of synthetic polymers such as polymethacrylate to create belts for nanodiscs is known in the art and described in, for example, Yasuhara, K., et al., Spontaneous Lipid Nanodisc Formation by Amphiphilic Polymethacrylate Copolymers, J Am Chem Soc. 2017 December 27; 139(51): 18657-18663, doi:10.1021/jacs.7b10591, the contents of which are incorporated herein by reference.

Lipid Bilayers of Nanodiscs

A variety of lipid molecules may be used to form the lipid bilayer of nanodiscs. In some embodiments, lipid molecules include phospholipids, such as phosphatidic acids, phosphoglycerides, and phosphosphingolipids. Phosphatidic acids include a phosphate group coupled to a glycerol group, which may be monoacylated or diacylated. Phosphoglycerides (or glycerophospholipids) include a phosphate group intermediate an organic group (e.g., choline, ethanolamine, serine, inositol) and a glycerol group, which may be monoacylated or diacylated. Phosphosphingolipids (or sphingomyelins) include a phosphate group intermediate an organic group (e.g., choline, ethanolamine) and a sphingosine (non-acylated) or ceramide (acylated) group. Phospholipids also includes salt (e.g., sodium, ammonium) of phospholipids. For phospholipids that include carbon-carbon double bonds, individual geometrical isomers (cis, trans) and mixtures of isomers are included.

Non-limiting examples of phospholipids include phosphatidylcholines, phosphatidylethanolamines, phosphatidylglycerols, phosphatidylserines, phosphatidylinositols, and phosphatidic acids, and their lysophosphatidyl (e.g., lysophosphatidylcholines and lysophosphatidylethanolamine) and diacyl phospholipid (e.g., diacylphosphatidylcholines, diacylphosphatidylethanolamines, diacylphosphatidylglycerols, diacylphosphatidylserines, diacylphosphatidylinositols, and diacylphosphatidic acids) counterparts.

In some embodiments, the acyl groups of the phospholipids are the same. In other embodiments, the acyl groups of the phospholipids are different. In some embodiments, the acyl groups are derived from fatty acids having C10-C24 carbon chains (e.g., acyl groups such as lauroyl, myristoyl, palmitoyl, stearoyl or oleoyl groups). Representative diacylphosphatidylcholines (i.e., 1,2-diacyl-sn-glycero-3-phosphocholines) include distearoylphosphatidylcholine (DSPC), dioleoylphosphatidylcholine (DOPC), dipalmitoylphosphatidylcholine (DPPC), dilinoleoylphosphatidylcholine DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoylstearoylphosphatidylcholine (PSPC), stearoylpalmitoylphosphatidylcholine (SPPC), and stearoyloleoylphosphatidylcholine (SOPC). Examples of diacylphosphatidylethanolamines (i.e., 1,2-diacyl-sn-glycero-3-phosphoethanolamines) include, but are not limited to, dioleoylphosphatidylethanolamine (DOPE), dipalmitoylphosphatidylethanolamine (DPPE), distearoylphosphatidylethanolamine (DSPE), dilauroylphosphatidylethanolamine (DLPE), dimyristoylphosphatidylethanolamine (DMPE), dierucoylphosphatidylethanolamine (DEPE), and palmitoyloleoylphosphatidylethanolamine (POPE).

Examples of diacylphosphatidylglycerols (i.e., 1,2-diacyl-sn-glycero-3-phosphoglycerols) include, but are not limited to, dioleoylphosphatidylglycerol (DOPG), dipalmitoylphosphatidylglycerol (DPPG), dierucoylphosphatidylglycerol (DEPG), dilauroylphosphatidylglycerol (DLPG), dimyristoylphosphatidylglycerol (DMPG), distearoylphosphatidylglycerol (DSPG), and palmitoyloleoylphosphatidylglycerol (POPG).

Examples of diacylphosphatidylserines (i.e., 1,2-diacyl-sn-glycero-3-phosphoserines) include, but are not limited to, dilauroylphosphatidylserine (DLPS), dioleoylphosphatidylserine (DOPS), dipalmitoylphosphatidylserine (DPPS), and distearoylphosphatidylserine (DSPS).

Examples of diacylphosphatidic acids (i.e., 1,2-diacyl-sn-glycero-3-phosphates) include, but are not limited to, dierucoylphosphatidic acid (DEPA), dilauroylphosphatidic acid (DLPA), dimyristoylphosphatidic acid (DMPA), dioleoylphosphatidic acid (DOPA), dipalmitoylphosphatidic acid (DPPA), and distearoylphosphatidic acid (DSPA). Examples of phospholipids include, but are not limited to, phosphosphingolipids such as ceramide phosphoryllipid, ceramide phosphorylcholine, and ceramide phosphorylethanolamine.

Nanodiscs may comprise one or more types of phospholipids. Thus, in some embodiments, a nanodisc may comprise two, three, four, or more, different types of phospholipids.

In some embodiments, a phospholipid is a native lipid extract. In some embodiments, a phospholipid is a headgroup-modified lipid, e.g., e.g., alkyl phosphates (e.g. 16:0 Monomethyl PE; 18:1 Monomethyl PE; 16:0 Dimethyl PE; 18:1 Dimethyl PE; 16:0 Phosphatidylmethanol; 18:1 Phosphatidylmethanol; 16:0 Phosphatidylethanol; 18:1 Phosphatidylethanol; 16:0-18:1 Phosphatidylethanol; 16:0 Phosphatidylpropanol; 18:1 Phosphatidylpropanol; 16:0 Phosphatidylbutanol; and 18:1 Phosphatidylbutanol); MRI imaging reagents (e.g. 14:0 PE-DTPA (Gd); DTPA-BSA (Gd); 16:0 PE-DTPA (Gd); 18:0 PE-DTPA (Gd); bis(14:0 PE)-DTPA (Gd); bis(16:0 PE)-DTPA (Gd); and bis(18:0 PE)-DTPA (Gd)); chelators (e.g., 14:0 PE-DTPA (Gd); DTPA-BSA (Gd); 16:0 PEDTPA (Gd); 18:0 PE-DTPA (Gd); bis(14:0 PE)-DTPA (Gd); bis(16:0 PE)-DTPA (Gd); and bis(18:0 PE)-DTPA (Gd)); glycosylated lipids (e.g., 18:1 Lactosyl PE); pH sensitive lipids (e.g., N-palmitoyl homocysteine; 18:1 DGS; 16:0 DGS; and DOBAQ); antigenic lipids (e.g., DNP PE and DNP Cap PE); adhesive lipids (e.g., 16:0 DG Galloyl); and functionalized lipids (e.g., 16:0 PA-PEGS-mannose; 16:0 Caproylamine PE; 18:1 Caproylamine PE; 16:0 MPB PE; 18:1 MPB PE; 16:0 Ptd Ethylene Glycol; 18:1 Ptd Ethylene Glycol; 16:0 Folate Cap PE; 16:0 Cyanur PE; 16:0 Biotinyl PE; 18:1 Biotinyl PE; 16:0 Biotinyl Cap PE; 16:0 Cyanur Cap PE; 18:1 Biotinyl Cap PE; 16:0 Dodecanoyl PE; 18:1 Dodecanyl PE; 16:0 Glutaryl PE; 18:1 Glutaryl PE; 16:0 Succinyl PE; 18:1 Succinyl PE; 16:0 PDP PE; 18:1 PDP PE; 16:0 Ptd Thioethanol; 18:1 Dodecanylamine PE; 16:0 Dodecanylamine PE; 16:0 PE MCC; 18:1 PE MCC; 16:0 hexynoyl PE; 16:0 azidocaproyl PE); sialic acid-modified lipid, or lipopolysaccharide (LPS).

Additional lipids that may be used as provided herein include lipids of the Archaea and other extremophilic microorganisms (see, e.g., de Rosa M. et al. Biosensors & Bioelectronics 9 (1994) 669-675, incorporated herein by reference). Lipids of the liver iron concentration originate from the formation of two or four ether links between two vicinal hydroxyl groups of a glycerol or more complex polyol, and C20, C25, or C40 isoprenoidic alcohols. Non-limiting examples of archaean-type lipids include those with archaeol (diether) and/or caldarchaeol (tetraether) core structures (Kaur G. et al. Drug Deliv 23(7) (2016) 2497-2512, incorporated herein by reference). In some embodiments, the lipids are extracted from the thermophilic archaeobacterium Sulfolobus solfatarius (Cavagnetto F et al. Biochimica et Biophysica Acta, 1106 (1992) 273-281, incorporated herein by reference).

### Compositions Containing Nanodiscs

The invention provides compositions that contain nanodiscs, such as those described above. The compositions include multiple nanodiscs. The compositions may include multiple nanodiscs that have the same or similar properties. Alternatively or additionally, the compositions may include nanodiscs that have different properties. For example and without limitation, the nanodiscs within a composition may vary in one or more of the following properties: size, shape, pathogen receptors, belt composition, or lipid bilayer composition.

The composition may include two or more sets of nanodiscs that differ in size. For example, the composition may include two, three, four, five, six, or more sets of nanodiscs that differ in size. The nanodiscs within each set may have substantially the same size, or they may have sizes that fall within a range. The nanodiscs within a set may have any size or range of sizes described above.

The composition may include two or more sets of nanodiscs that differ in shape. For example, the composition may include two, three, four, five, six, or more sets of nanodiscs that differ in shape. The nanodiscs within each set may have substantially the same shape. The nanodiscs within a set may have any shape described above.

The composition may include two or more sets of nanodiscs that have different pathogen receptors. For example, the composition may include two, three, four, five, six, or more sets of nanodiscs that have different pathogen receptors. The nanodiscs in different sets may have receptors to different pathogens. The nanodiscs in different set may have different receptors for the same pathogen. For example, as described above, many viruses bind to more than one molecule on the surface of a target cell, and each molecule or a portion of each molecule may be contained on nanodiscs from different sets. The nanodiscs in one or more of the sets may contain multiple receptors for the same pathogen, as described above.

The composition may include two or more sets of nanodiscs that differ in belt composition. For example, the composition may include two, three, four, five, six, or more sets of nanodiscs that differ in belt composition. The sets may have belts of different types of molecules, e.g., nucleic acid vs. protein, of the sets may have different belt molecules, e.g., different proteins, proteins of different size, etc.

The composition may include two or more sets of nanodiscs that differ in composition of the lipid bilayer. For example, the composition may include two, three, four, five, six, or more sets of nanodiscs that differ in composition of the lipid bilayer.

Compositions containing different sets of nanodiscs may contain the nanodiscs of the different sets in the same amount or quantity, or they may contain nanodiscs of the different sets in different amounts or quantities. For example and without limitation, a composition may contain nanodiscs in two different sets ata ratio of 1:1, 1:1.5, 1:2, 1:3, 1:4, 1:5, 1:6, 1:8, or 1:10. The ratio may be a stoichiometric ratio, i.e., a ratio of the number of individual discs, or it may be a mass ratio, i.e., a ratio of the total mass of discs in one set to the total mass of discs in another set.

The compositions may be formulated for administration to a subject. For example and without limitation, the composition may be formulated for oral, intravenous, enteral, parenteral, dermal, buccal, topical (including transdermal), injection, intravenous, nasal, or pulmonary administration. Preferably, the compositions are formulated for intravenous injection.

The compositions may be in the form of a sterile injectable aqueous solution or suspension. This suspension may be formulated according to the known art using those suitable dispersing or wetting agents and suspending agents. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

### Use of Nanodisc-Containing Compositions to Treat or Prevent Infections

The invention provides methods of treating or preventing pathogen infections by providing compositions containing pathogen-binding nanodisc compositions, such as those described above, to a subject. The subject may have a pathogen infection or may be at risk of contracting a pathogen infection.

FIG. 2 illustrates a mechanism of extracellular neutralization of virus by nanodiscs according to an embodiment of the invention. Nanodiscs circulating in the blood and interstitial fluids bind to virus particles. The size of the nanodiscs and their functionalization with multiple copies of receptor for the virus allows the nanodiscs to aggregate virus particles. The aggregated virus particles are phagocytosed by macrophages and thereby neutralized.

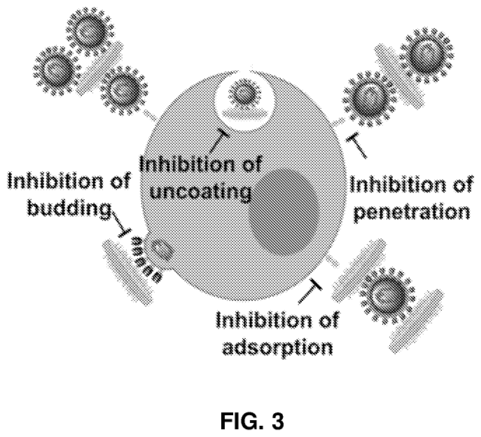

FIG. 3 illustrates additional mechanisms of neutralization of virus by nanodiscs according to embodiments of the invention. In one mechanism, nanodiscs promote aggregation of extracellular virus particles, thereby preventing the particles from adsorbing or attaching to their natural receptors on the surface of a target cell. In another mechanism, nanodisc-bound virus particles bind to natural receptors of the surface of a target cell but are prevented from penetrating or entering the target cell. In another mechanism, nanodisc-bound virus particles enter target cells but are prevented from uncoating inside the target cell. In another mechanism, nascent virus particles form at the surface of a target cell, but binding of nanodiscs to viral markers, such as proteins, on the surface of the target cell prevents the nascent virus particles from budding and exiting from the target cell.

In another mechanism by which compositions of the invention neutralize viruses, the nanodiscs act as decoys that resemble the plasma membrane of a target cell. A virus particle binds to the receptor on a nanodisc and injects its nucleic acid genome across the lipid bilayer. In the context of infection of a target cell, the nucleic acid would then either be transcribed, reverse transcribed, integrated, or replicated within the host cell. However, after binding to a nanodisc, the nucleic acid merely gets transported to the other side of the lipid bilayer and into the extracellular milieu in which the virus particle and nanodisc are suspended. Thus, the viral genome is unable to express its genes or reproduce itself, and the virus is neutralized. Details of ejection of viral nucleic acids are described in Nasr, M. L., et al., Covalently circularized nanodiscs for studying membrane proteins and viral entry, Nat Methods, 2017 January, 14(1): 49-52, doi:10.1038/nmeth.4079, the contents of which are incorporated herein by reference.

Both the size of the nanodiscs and the presence of multiple copies of pathogen receptors on the nanodiscs contribute to the efficacy of the compositions at treating and preventing infections. The nanodiscs are large enough to bind multiple copies of pathogen particles, such as virus particles or bacterial cells, which allows them to promote aggregation. The same properties help block process of virus replication, such as adsorption, penetration, uncoating, and budding, at or within a target cell.

Providing the composition may include administering the composition to a subject. The composition may be administered by any suitable route of administration. For example and without limitation, the composition may be formulated for oral, intravenous, enteral, parenteral, dermal, buccal, topical (including transdermal), injection, intravenous, nasal, or pulmonary administration. Preferably, the compositions are formulated for intravenous injection.

The compositions are may be used to combat infection from any type of pathogen. The pathogen may be any type of microorganism. For example and without limitation, the pathogen may be a bacterium, virus, fungus, or plasmodium.

As indicated above, the compositions of the above may contain multiple sets of nanodiscs that are specific to different pathogens. Thus, the methods of the invention are useful to treat or prevent infection from multiple pathogens simultaneously.

### Use of Nanodisc-Containing Compositions to Disinfect Aqueous Media

The invention also provides methods of disinfecting aqueous media using compositions of the invention. As described above, the nanodiscs of the invention disable pathogens via a variety of mechanisms, including aggregation, prevention of replication in target cells, and acting as target cell decoys. Thus, the compositions are effective at neutralizing pathogens outside the body.

The methods of disinfection involve providing compositions of the invention, such as those described above, to an aqueous medium. The aqueous medium may be any aqueous fluid that may contain a pathogen and may come into contact with a subject or target cells that could become infected by the pathogen. For example and without limitation, the aqueous medium may be a foodstuff, beverage, biological fluid sample, medical reagent, or research reagent. The biological fluid may be blood, plasma, serum, semen, sputum, saliva, or milk. The aqueous medium may be a solution or suspension that will be contacted with, or transferred into, the body of a subject. The aqueous medium may be synthetic growth medium

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of treating or preventing a pathogen infection in a subject, the method comprising:
providing to a subject a composition comprising a plurality of nanodiscs, each nanodisc being functionalized with a plurality of receptors that specifically bind a pathogen, wherein the plurality of nanodiscs specifically bind the pathogen and/or a pathogen-specific marker on an infected cell to prevent the pathogen from interacting with a target cell and/or inhibit one or more processes associated with pathogen replication in the target cell, thereby treating or preventing a pathogen infection in a subject.

2. The method of claim 1, wherein the plurality of nanodiscs circulate in blood of the subject and aggregate pathogen via the pathogen binding to one or more of the plurality of nanodiscs, thereby reducing a number of circulating pathogens.

3. The method of claim 2, wherein aggregation of the pathogen facilitates or induces phagocytosis of the pathogen, thereby preventing the pathogen from interacting with the target cell.

4. The method of claim 1, wherein the pathogen is a virus.

5. The method of claim 4, wherein binding of one or more of the plurality of nanodiscs to the pathogen inhibits one or more virus replication steps selected from the group consisting of attachment or adsorption of the virus to a cell surface of the target cell, penetration of the virus into the target cell, and uncoating of the virus within the target cell.

6. The method of claim 4, wherein the plurality of nanodiscs bind pathogen-specific marker on a cell surface of the infected cell, thereby inhibiting budding of the virus from the infected cell.

7. The method of claim 1, wherein the plurality of nanodiscs are of different sizes.

8. The method of claim 7, wherein each nanodisc of the plurality of nanodiscs comprises a diameter of from about 6 nm to about 100 nm.

9. The method of claim 1, wherein the plurality of nanodiscs are of the same size.

10. The method of claim 1, wherein the plurality of receptors comprise at least two different sets of receptors, the receptors in different sets being specific for different pathogens.

11. The method of claim 1, wherein the plurality of receptors are a plurality of the same receptor, present in multiple copies on each nanodisc.

12. The method of claim 1, wherein the plurality of nanodiscs comprise a first set of nanodiscs and a second set of nanodiscs, wherein the first set of nanodiscs comprises a pathogen receptor that binds to a first pathogen and the second set of nanodiscs comprises a second pathogen receptor that binds to a second pathogen that is different from the first pathogen.

13. The method of claim 12, wherein the first set of nanodiscs and the second set of nanodiscs are present in different amounts.

14. A composition comprising a plurality of nanodiscs, each nanodisc being functionalized with a plurality of receptors that specifically bind a pathogen.

15. The composition of claim 14, wherein the plurality of nanodiscs are of different sizes.

16. The composition of claim 15, wherein each nanodisc of the plurality of nanodiscs comprises a diameter of from about 6 nm to about 100 nm.

17. The composition of claim 14, wherein the plurality of nanodiscs are of the same size.

18. The composition of claim 14, wherein the plurality of receptors are a plurality of the same receptor, present in multiple copies on each nanodisc.

19. The composition of claim 14, wherein the plurality of receptors comprise at least two different sets of receptors, the receptors in different sets being specific for different pathogens.

20. The composition of claim 14, wherein the plurality of nanodiscs comprise a first set of nanodiscs and a second set of nanodiscs, wherein the first set of nanodiscs comprises a pathogen receptor that binds to a first pathogen and the second set of nanodiscs comprises a second pathogen receptor that binds to a second pathogen that is different from the first pathogen.

21. The composition of claim 20, wherein the first set of nanodiscs and the second set of nanodiscs are present in different amounts.

22-26. (canceled)
